(19) United States
(12) Patent Application Publication
(10) Pub. No.: US 2008/0011291 A1
(43) Pub. Date: Jan. 17, 2008

(54) Inhaler Valve

(76) Inventors: Ian Fletcher, Leicestershire (GB); Stephen Metcalf, Leicestershrie (GB); Darren Hodson, Leicestershire (GB)

Correspondence Address: MORGAN LEWIS & BOCKIUS LLP, 1111 PENNSYLVANIA AVENUE NW, WASHINGTON, DC, 20004, US

(21) Appl. No.: 11/664,739

(22) PCT Filed: Oct. 6, 2005
(86) PCT No.: PCT/SE05/01474

§ 371 Date: Apr. 5, 2007

(30) Foreign Application Priority Data
Oct. 8, 2004 (SE) 0402434-5

(51) Int. Cl. A61M 11/00

(52) U.S. Cl. 128/200.14

(57) ABSTRACT

Inhaler valve (30) comprising a gathering ring (70) comprised of a non-elastomeric polymer-material, wherein at least one annular section (110, 140, 160, 180) of the gathering ring (70) is formed to be deformable in the radial direction in order to absorb over compression of the ring outer periphery when crimping the valve (30) onto a container (20). There is also provided an inhaler container (10) with such an inhaler valve.

The present invention relates to the art of inhaler devices, and in particular to an inhaler can valve.

### BACKGROUND OF THE INVENTION

Many types of drugs are provided in fluid form, such as a solution or suspension or emulsion of drug in a propellant, and are adapted for oral inhalation by a patient. As one example, a container might contain asthma medicine such as fluticasone propionate. During a typical manufacturing process, the container is sealed by crimping a metering valve onto the neck of the container. The container is then charged through the valve with the propellant based drug product.

In order to deliver the drug to the patient, the container operates in conjunction with an actuator as a system commonly known as a metered dose inhaler (MDI) system. The actuator includes a housing having an open container-loading end and an open mouthpiece. A nozzle element is disposed within the housing and includes a valve stem communicating with a nozzle orifice. The orifice is aimed toward the mouthpiece. In order to receive a properly metered dosage of medicine from the container, the patient installs the container into the actuator through the container-loading end until the valve stem is fitted into the receiving bore of the nozzle element. With the container so installed, the opposite end of the container typically extends to some degree outside the actuator housing. The patient then places the mouthpiece into his or her mouth and pushes downwardly on the exposed container end. This action causes the container to displace downwardly with respect to the valve stem, which in turn actuates the valve. Owing to the design of the valve, the design of the nozzle element, and the pressure differential between the interior of the container and the ambient air, a short burst of precisely metered, atomized formulation is thereby delivered to the patient.

FIG. 1 shows a sectional view of one embodiment of an inhaler container 10. The inhaler 10 is comprised of a can 20 and a valve assembly 30. Due to the relatively high pressure of the propellant, the valve assembly must be firmly attached to the can 20. FIG. 2 shows the can 20 and the valve assembly 30 before they are attached to each other. The valve assembly is basically comprised of a valve mechanism 40 with a valve body 90 and a valve stem 100, a gasket 50, a ferrule 60, and a support ring 70. Further, there is an opening 130 in the valve body 90, through which the drug enters the valve. In FIG. 1 and all following figures, the inhaler container 10 is shown in the operating position, i.e. with the valve directed downwards. As can be seen in FIG. 1 the valve assembly 30 is attached to the container 20 by a crimp 80, i.e. the upper section of the ferrule 60 is crimped in a crimping apparatus so that it closely clasps the lower section of the container 20. Further, the inhaler can 10 is sealed by the upper edge of the container 20 being pressed against the gasket 50 by the crimp 80.

The gathering ring 70 is designed to reduce product ullage and provide a defined end of life dosing. This is achieved by forming the gathering ring 70 with an inclined area towards its internal diameter, so that it gathers and guides the drug product in the container close to the opening 130 in the valve body 100. The gathering ring 70 is retained in position by a dimensional interference fit between its internal diameter (ID) and the external diameter of the valve body 90 which houses the valve stem 100. When the valve is crimped onto the pMDI can, the clearance between the outer diameter of the ring and the inner diameter of the can is small (FIG. 1). Further, the gathering ring 70 is made of a pharmacologically inert and propellant resistant polymer with respect to the contents in the container, and reduces the contents contact with the gasket 50, which may not be chemically inert to the same extent. Examples of such pharmacologically inert and propellant resistant polymers are acetal, polyamide (e.g. Nylon®), polycarbonate, polyester, fluorocarbon polymer (e.g. Teflon®), polyethylene, polybutylterephthalate (PBT) or the like. One gathering ring of this type is disclosed in U.S. Pat. No. 4,349,135.

WO 94/29192 discloses a gathering ring that is integrated with the gasket as one integral component of elastomer material. However, there is increased potential for extractives from the elastomer contaminating the drug product, compared to the non elastomer polymers above.

As the valve 10 is crimped onto the can to the container 20 there is a potential risk that the gathering ring 70 is compressed causing a reduction in the gathering ring internal diameter (ID) and increased interference with the body 90 of the valve 30 (FIG. 1). Where the reduction in the gathering ring ID is sufficiently large, for example when the valve 30 is crimped 80 particularly tightly, as is the case illustrated in FIG. 1, performance of the valve is detrimentally affected through restriction of valve stem 100 movement in the valve body 90. This can result in increased actuation force, an increase in actuation weight variability or in the extreme, complete jamming of the valve 30. Potential reduction of the gathering ring ID and subsequent interference with valve stem movement is further increased by exposure to elevated temperatures eg. during leakage stress testing, which can soften the polymeric components of the valve 30 and allow greater movement under residual crimping forces.

In order to detect potentially compromised valves 30, a number of crimp 80 measurement methods have been developed, but such measurements require an extra step in the production of inhaler containers 10 and thus also involves additional costs. Plus they are an indirect measure of the key parameter (ID) and not wholly reliable as a predictor of whether or not valve jamming is likely to occur.

### SUMMARY OF THE INVENTION

The object of the invention is to provide a new inhaler valve, which overcomes one or more drawbacks of the prior art. This is achieved by the inhaler valve as defined in claim 1.

One advantage with such an inhaler valve is that it prevents over-compression of the gathering ring and valve body and provides a valve which is robust with respect to crimping and exposure to heat.

Another advantage is that the inhaler valve can be crimped tightly to the can without impacting on performance thereby accommodating variability within the crimping process and component dimensions and physical properties.

Another advantage is that measurements of crimp diameter can be omitted without risk of defective valve mechanisms due to over crimping.

Still another advantage is that the tight crimp prevents contact of the contained product with the elastomeric sealing gasket.

Embodiments of the invention are defined in the dependent claims.

### BRIEF DESCRIPTION OF THE DRAWINGS

The invention will be described in detail below with reference to the drawings, in which

FIG. 1 schematically shows a cross sectional view of an inhaler can for containing a pharmaceutical substance in a pressurized propellant to be included in an inhalation device.

FIG. 2 shows the inhaler container of FIG. 1 in an unassembled state.

FIG. 3 schematically shows a cross sectional view of an inhaler can in an unassembled state, the can comprising a valve according to one embodiment of the present invention.

FIG. 4 shows the inhaler can of FIG. 3 in an assembled state.

FIG. 5 schematically shows a cross sectional view of an inhaler can in an unassembled state, the can comprising a valve according to one embodiment of the present invention.

FIG. 6 shows the inhaler can of FIG. 5 in an assembled state.

FIG. 7 schematically shows a cross sectional view of an inhaler can in an unassembled state, the can comprising a valve according to one embodiment of the present invention.

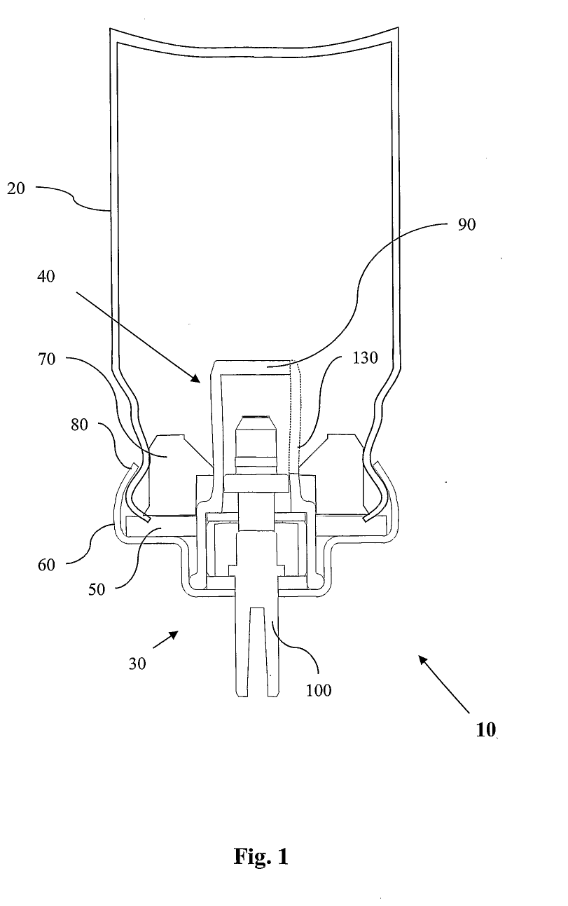

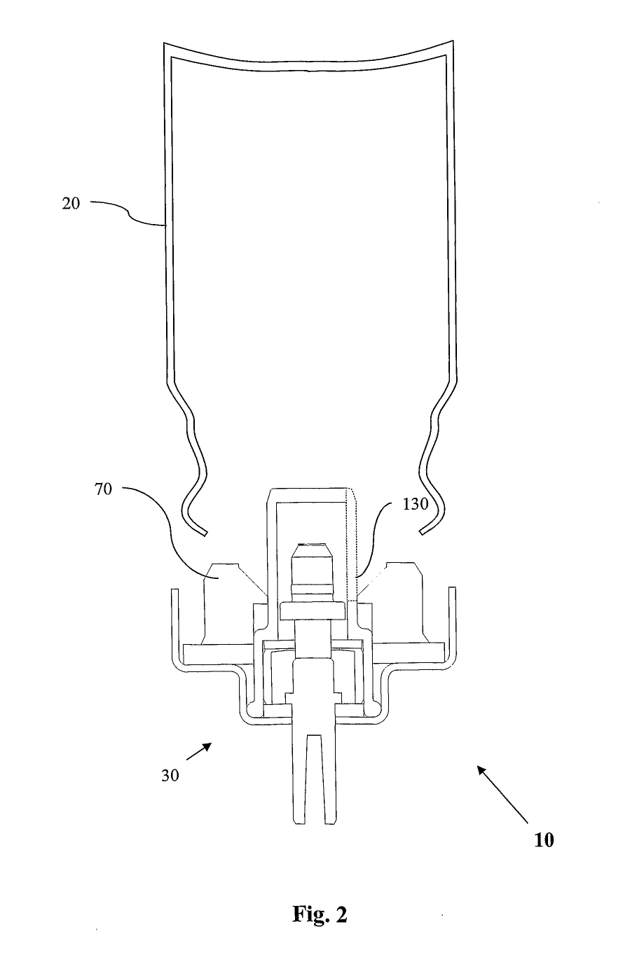

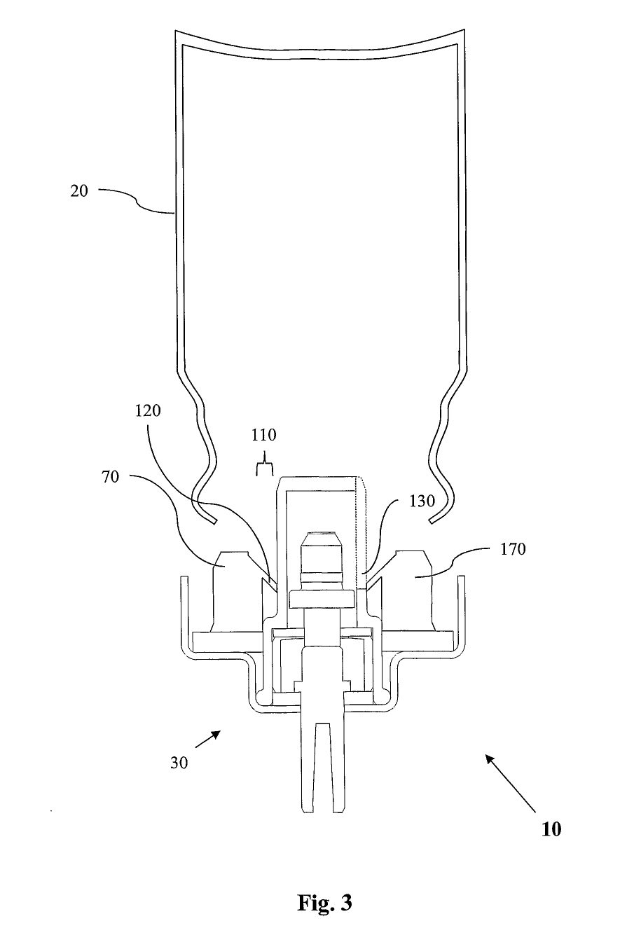

### DETAILED DESCRIPTION OF PREFERRED EMBODIMENTS

In order to avoid the above problems, related to radial compression of the gathering ring 70, when the valve is crimped to the container 20, the present invention provides a new gathering ring 70 of an inert non-elastomeric polymer-material, the gathering ring 70 comprising at least one annular section formed to be deformable in the radial direction. In the context of the present invention, the term deformable annular section refers to a section of the gathering ring that is deformed by a force that is less than the force required to deform the main section 170 of the gathering ring 70. Preferably, the deformable annular section is located at the inner and/or outer periphery of the gathering ring. By providing such a deformable annular section, the compressive force associated with crimping is absorbed by deformation of said deformable annular section. The deformation of the deformable annular section can be both elastic and/or plastic.

FIG. 3 shows one embodiment of the present invention, wherein a deformable annular section 110 is provided at the inner periphery of the gathering ring 70. In this embodiment the deformable annular section 110 is an inclined flange 120, shaped as a thin-walled truncated cone, which acts as a flexible member that deforms under crimp forces without applying an excessive force on the valve body 90. The flange 120, can be formed in any suitable way, provided that it exhibits the desired deformability, but it is preferably formed so that it guides the content in the can to the valve inlet opening 130. In this embodiment, the main section 170 of the gathering ring 70 will be deformed by the compressive force resulting from a tight crimp, but due to the deformability of the flange 120 the compressive force is not transferred to the valve body 90.

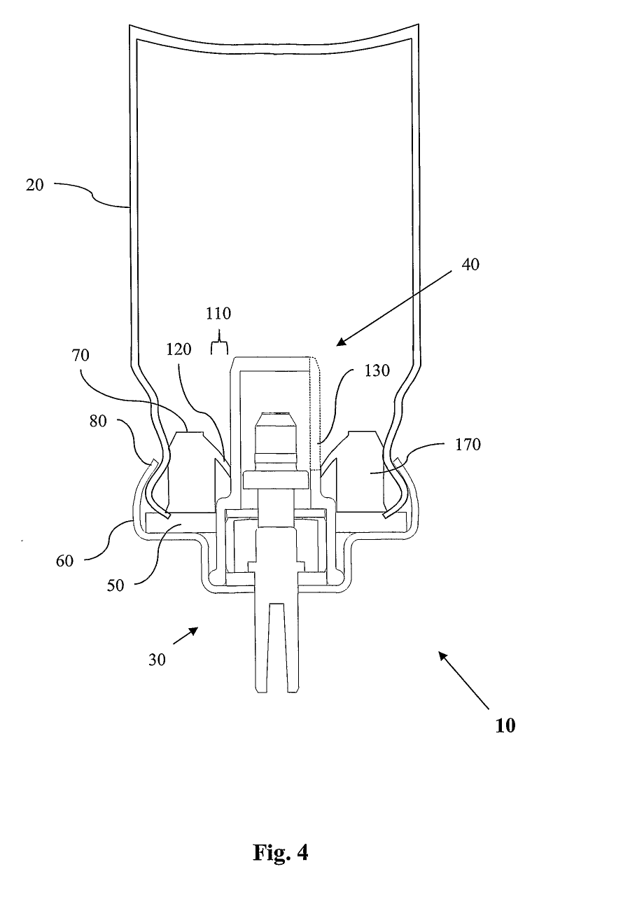

FIG. 4 illustrates the valve according to FIG. 3 crimped onto a can, using a tight crimp. The flange 120 is shown in a deformed state. The shape of the flange 120 determines the amount of pressure that the gathering ring 70 applies on the valve body 90. One way to adjust the resulting pressure is to control the thickness of the flange 120, whereby a thinner flange 120 gives a lower pressure (see verifying example below).

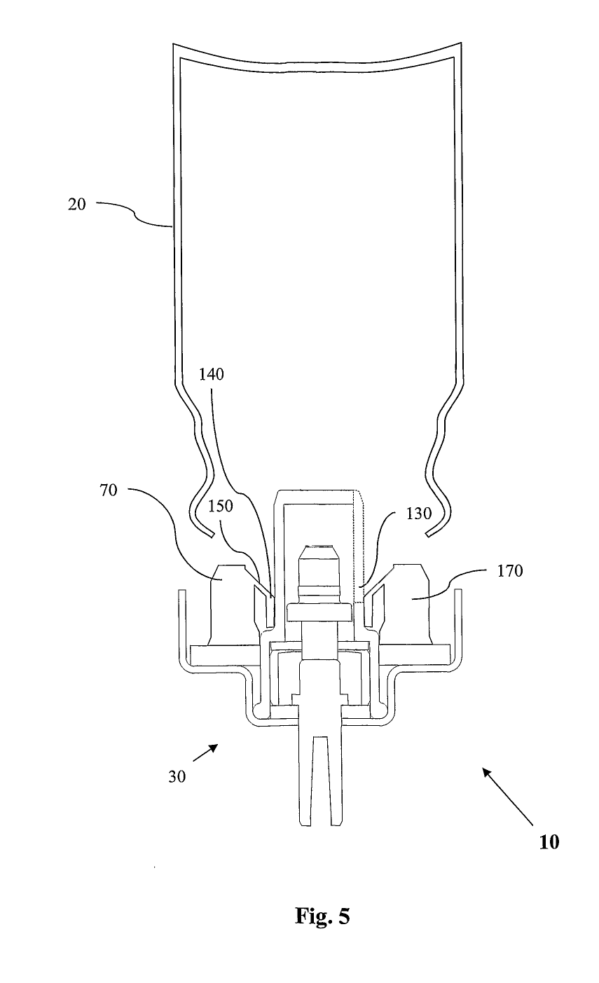

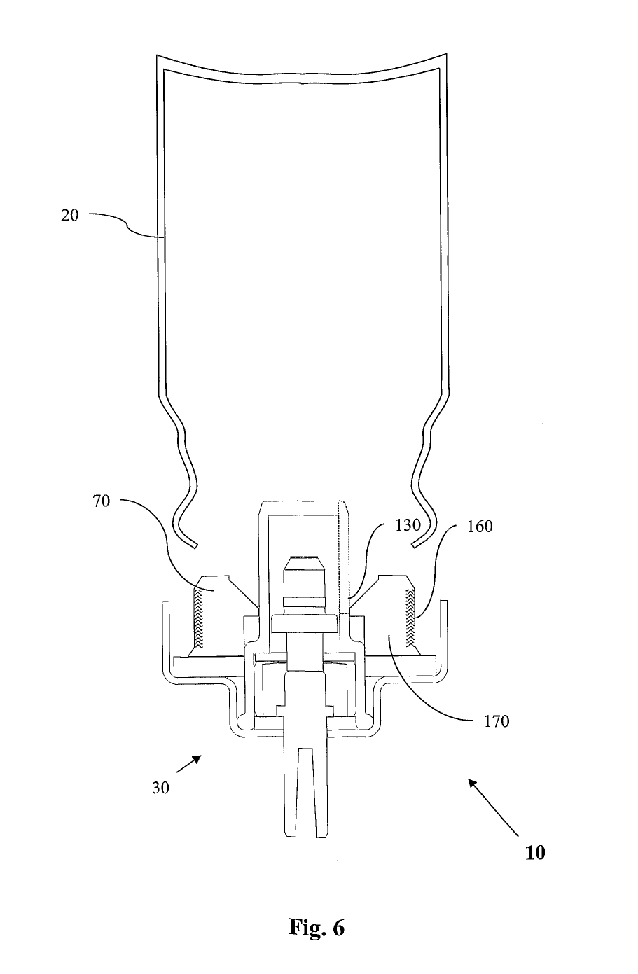

Further, the gathering ring 70 can be designed to apply an essentially constant pressure on the valve body 90, irrespective of the magnitude of compression pressure applied on the gathering ring 70 by the crimp 80. Generally this can be achieved by a gathering ring 70 wherein the deformable annular section is located at an intermediate position between an inner rigid section and an outer rigid section 170. FIG. 5 shows one example of such a design, comprising a rigid inner ring 140 in addition to a flexible flange section 150. In this embodiment, the flexible flange section 150 absorbs the deformation, while the inner ring 150 remains essentially unaffected by the crimp 80 compression.

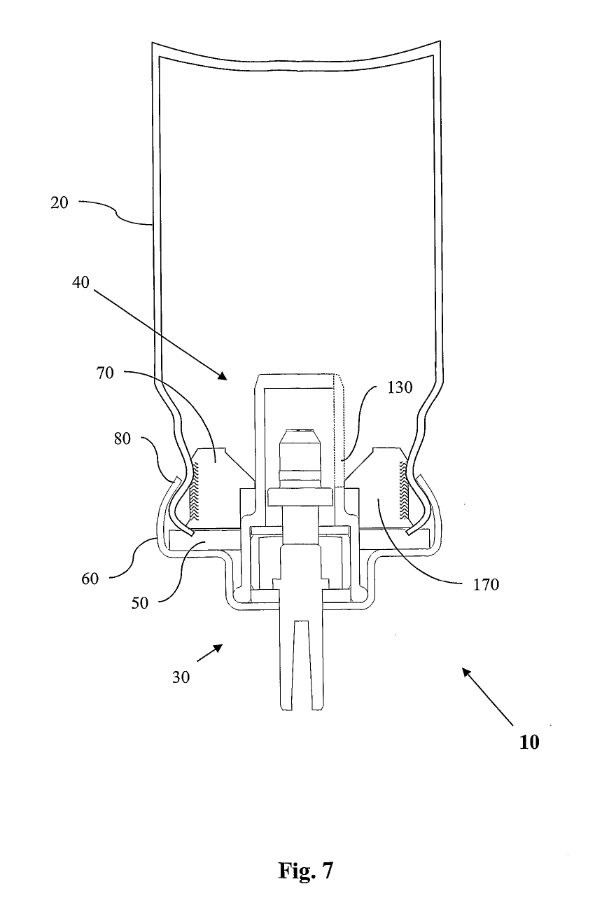

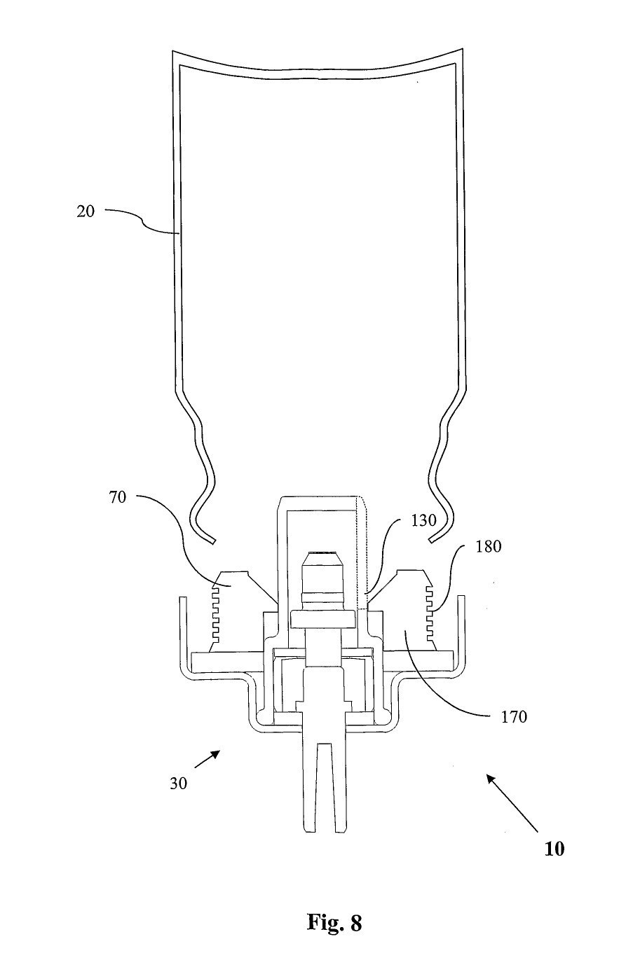

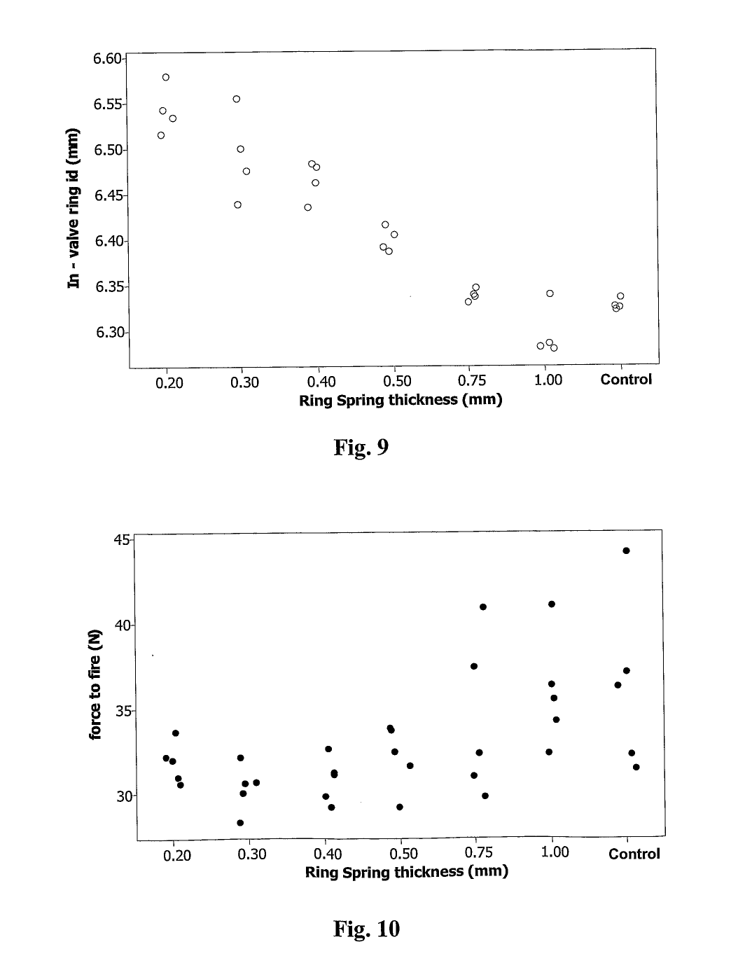

FIGS. 6 and 7 shows another embodiment of the present invention, wherein the deformable annular section 160 is located at the outer periphery of the gathering ring. In this embodiment, the material properties have been altered for the deformable annular section 160, in such a way that it is more easily deformed compared with the main section 170 of the gathering ring 70. The altered material properties can be achieved in a number of ways, such as providing the outer rim of the ring as a foamed polymer, formed in-situ in the molding process or added thereto after molding, or providing the outer rim of the ring as an elastomeric material, or providing an internal cavity in the vicinity of the gathering ring outer periphery, leaving a thin flexible outer peripheral wall, etc. As is shown in FIG. 7, the compression of the crimped neck results in a local compression of the deformable annular section 160, which compression force is not transferred to the main section 170 and thus not to the inner rim of the gathering ring 70.

FIG. 8 shows still another embodiment of the present invention, wherein the deformable annular section 180 is located at the outer periphery of the gathering ring 70. In this embodiment, the structure of the gathering ring outer peripheral surface has been altered in so that it is more easily deformed in the radial direction. In the disclosed embodiment, a number of circumferential grooves are formed in the gathering ring outer periphery. The circumferential grooves in turn defines a number of circumferential deformation ridges 180, and by giving the ridges 180 a suitable width, the deformability of the deformable annular section 180 can be controlled. As the valve is crimped to the can, these ridges 180 are preferentially deformed such that the horizontal forces are not transferred through the main section 170 to the internal diameter of the gathering ring 70.

The proposed approach using at least one deformable annular section 110, 140, 160, 180 to accommodate for over-crimping, changes the current tolerance design to a significantly more robust parameter design, the performance of which is unaffected by over compression during crimping and subsequent exposure to heat.

Verifying Experiments:

FIGS. 9 and 10 shows results from tests using gathering rings 70 of the type disclosed in FIGS. 3 and 4. A number of gathering rings 70 with different thicknesses of the flange 120 were fitted to the valve body and crimped tightly onto a can and the resulting inner diameter (ID) and actuation force for the valve was registered. The results are shown in FIGS. 9 and 10 respectively. The results confirm reduced potential for constriction of the ring inner diameter and no increase in actuation force with flange-thicknesses less than 0.5 mm.

1. Inhaler valve (30) comprising a gathering ring (70) comprised of an inert non-elastomeric polymer-material, characterized in that at least one annular section (110, 140, 160, 180) of the gathering ring (70) is formed to be deformable in the radial direction.

2. Inhaler valve (30) according to claim 1 characterized in that the deformable annular section (110) is located at the inner periphery of the gathering ring (70).

3. Inhaler valve (30) according to claim 1 characterized in that the deformable annular section (160, 180) is located at the outer periphery of the gathering ring (70).

4. Inhaler valve (30) according to claim 1 characterized in that the deformable annular section (140) is located at an intermediate position between an inner rigid section (150) and an outer rigid section (170).

5. Inhaler valve (30) according to claim 2 characterized in that the deformable annular section (110) is an inclined flange (120).

6. Inhaler valve (30) according to claim 3 characterized in that the deformable annular section is formed by a number of circumferential grooves (180) in the outer periphery of the gathering ring (70).

7. Inhaler valve (30) according to claim 3 characterized in that the deformable annular section is formed by a foamed section (160) in the outer periphery of the gathering ring (70).

8. Inhaler valve (30) according to claim 4 characterized in that the intermediate deformable annular section is formed by a thin-walled radial section (140) of the gathering ring.

9. Inhaler valve (30) according to any of the preceding claims characterized in that the gathering ring is made of a material or combination of the materials in the group: acetal, polyamide, polycarbonate, polyester, fluorocarbon polymer, polybutylterephthalate and polyethylene.

10. Inhaler container (10) characterized in that it comprises an inhaler valve (30) according to any of the claims 1 to 9.
